(19) United States
(12) Patent Application Publication
(10) Pub. No.: US 2005/0283215 A1
(43) Pub. Date: Dec. 22, 2005

(54) Electrode needle

(76) Inventors: Kai Desinger, Berlin (DE); Andre Roggan, Berlin (DE); Markus Fay, Berlin (DE); Rainer Rothe, Lichtenau (DE)

Correspondence Address: BECK AND TYSVER P.L.L.C., 2900 THOMAS AVENUE SOUTH, SUITE 100, MINNEAPOLIS, MN, 55416, US

(21) Appl. No.: 10/518,196

(22) PCT Filed: Jun. 17, 2003
(86) PCT No.: PCT/EP03/06393

§ 371 Date: Jul. 25, 2005

(30) Foreign Application Priority Data
Jun. 19, 2002 (DE) 102 28 085.1

(51) Int. Cl. A61N 1/05

(52) U.S. Cl. 607/116

(57) ABSTRACT

The electrode needle according to the invention has a shaft 4 and at least one active electrode 7 and is distinguished in that the shaft 4 includes a nuclear magnetic resonance-active marker element 15 spatially associated with the active electrode 7. The nuclear magnetic resonance-active marker element can contain ferromagnetic material such as for example iron, cobalt, nickel or steel.

This application is a continuation of and claims priority to PCT/EP03/06393, filed Jun. 17, 2003 and claims priority thereto. In addition, this application claims priority to German patent application 102 28 085.1, filed Jun. 19, 2002.

### FIELD OF THE INVENTION

The invention concerns an electrode needle comprising a shaft and at least one electrode provided on the shaft.

### BACKGROUND OF THE INVENTION

A method of treating pathologically altered body tissue, which is known in medicine, is electrosurgical and in particular electrothermal sclerosing of the tissue in question. That method is of particular interest for the therapy of organ tumors, for example liver tumors. To perform the sclerosing procedure one or more electrodes are placed in the tissue to be sclerosed, that is to say the tumor tissue, or in the immediate proximity thereof, and an alternating current is caused to flow between the electrodes or an electrode and a so-called neutral electrode which is fixed externally to the body. When the current flows between the electrode and the neutral electrode (possibly also between a plurality of electrodes and one or more neutral electrodes), that is referred to as a monopolar electrode arrangement. If in contrast the current flows between the electrodes themselves disposed in the tissue (in that case at least two electrodes have to be introduced into the tissue), that is referred to as a bipolar arrangement. The electrode provided for placement in the tissue is generally arranged on an electrode needle.

To cause sclerosing of the pathologically altered tissue, a current flow is induced by means of a high frequency generator between the so-called active electrodes which are in electrically conductive contact with the body tissue, and for example a neutral electrode. In that situation the electrical resistance of the body tissue provides that the alternating current is converted into heat. At temperatures of between 50° C. and 100° C. that involves massive denaturing of the body-specific proteins and consequently causes the tissue area involved to die. By virtue of the high current density in the region of the active electrodes heating of the tissue takes place predominantly where the active electrodes are in electrically conductive contact with the body tissue.

In the interests of effective treatment it is advantageous to check the progress of the treatment in as near real-time relationship as possible. For that purpose doctors are going over to monitoring sclerosing of tumor tissue by the application of high frequency current by means of nuclear magnetic resonance tomography. In that respect, in a nuclear magnetic resonance tomography recording, it is possible to see not only the differences between healthy tissue and tumor tissue, but also between sclerosed and non-sclerosed tissue.

For effective sclerosing of tumor tissue however precise placement of the active electrodes arranged on an electrode needle, in the tissue to be sclerosed or in the proximity thereof, is also important.

### SUMMARY OF THE INVENTION

Therefore the object of the invention is to provide an electrode needle which permits reliable and accurate placement of the active electrodes in the body.

That object is attained by an electrode needle as set forth in claim 1. The appendant claims set forth further advantageous configurations of the electrode needle according to the invention.

The electrode needle according to the invention has a shaft and at least one active electrode and is distinguished in that the shaft includes a nuclear magnetic resonance-active marker element which is spatially associated with the active electrode. The nuclear magnetic resonance-active marker element is composed of a material whose magnetic properties differ both from those of the shaft and electrode material and also from those of the body tissue which is substantially composed of water. In accordance with the invention that can be achieved in that materials with paramagnetic properties (for example bronze, aluminum, copper, brass) or ferromagnetic properties (for example iron, nickel, steel) or alloys thereof are used.

The invention is based on the following idea:

Conventional electrode needles are to be of a body-compatible configuration in the form of treatment devices which are to be brought into contact with the body tissue. Therefore the shaft comprises body-compatible materials, for example body-compatible plastic materials or body-compatible metals which are possibly covered with body-compatible plastic materials. The active electrodes are made from metal and are either formed by a part of the shaft or are integrated thereinto. A material which is frequently used for the active electrodes or for shafts because of its good body compatibility on the one hand and good nuclear magnetic resonance compatibility on the other hand is titanium or alloys thereof. By virtue of suitable artefacts that structure guarantees good imaging and representation of the entire electrode needle.

An aspect of crucial significance for good treatment success however is optimum positioning of the active region of the electrode needle, that is to say the region which is in electrically conducting contact with the surrounding body tissue and in the surroundings of which the therapeutic, that is to say coagulative action occurs due to the high current density. In the case of electrode needles in accordance with the state of the art, that region cannot be distinguished from the remaining part of the needle in the magnetic resonance-tomographic recording so that the position of the needle relative to the pathological tissue (for example a tumor) can only be determined with difficulty.

If in contrast the active electrodes of the electrode needle are marked with a nuclear magnetic resonance-active marker element, then the nuclear magnetic resonance-active marker element leaves behind in the nuclear magnetic resonance-tomographic recording artefacts which make the position of the active electrodes visible. As the tissue to be sclerosed, for example a tumor tissue, stands out from the healthy tissue in the recording, monitoring of placement of the active electrodes, which is to be implemented for the treatment, is possible by means of the electrode needle according to the invention.

In order to make not just the position of the active electrode of electrode needle visible in the nuclear magnetic resonance-tomographic recording, but also the extent thereof, in a configuration of the electrode needle the nuclear magnetic resonance-active marker element extends over the entire axial length of the active electrode. Alternatively the nuclear magnetic resonance-active marker element can extend over the entire axial length of the shaft of the electrode needle with the exception of the axial length of the active electrode so that in the nuclear magnetic resonance-tomographic recording that involves an artefact in which the region of the active electrode is cut out. That gives so-to-speak a “negative image” of the active electrodes.

In an embodiment of the electrode needle the nuclear magnetic resonance-active marker element is in the form of a wire. Nuclear magnetic resonance-active marker elements in wire form are inexpensive to produce and easy to handle. They can comprise a simple wire containing ferromagnetic material.

In a configuration of the embodiment the electrode needle has a shaft with a lumen. The wire is arranged in the interior of the lumen, which is not in contact with the body tissue. In this embodiment there is no need to select a material which involves good body compatibility as the nuclear magnetic resonance-active material, which increases the number of materials suitable for use for the electrode needle according to the invention. The wire can be fixed for example to the inside of the casing of the shaft, which surrounds the lumen. An electrode needle of such a configuration is simple and inexpensive to produce.

In an alternative embodiment of the electrode needle according to the invention the nuclear magnetic resonance-active marker element is in the form of a coating. The coating can include for example ferromagnetic material. This embodiment in the form of a coating makes it possible for the entire surface of the active electrode to be marked in a simple manner. In addition the coating can be kept very thin so that the amount of space required for the nuclear magnetic resonance-active marker element is slight. The coating is therefore particularly suitable for very thin electrode needles.

In a configuration of this embodiment the electrode needle has a shaft with a casing surrounding a lumen, wherein the coating is applied to the inside surface of the casing. As the coating can be thin it takes up no space which is provided for other components of the electrode needle such as for example the electrical feed line to the active electrode and/or a coolant feed line for cooling the active electrode.

In another alternative configuration of the embodiment the active electrode encloses an axial portion of the shaft. In this configuration the coating is disposed between the shaft and the active electrode either on the shaft or on the electrode.

Instead of being in the form of a coating or a wire the nuclear magnetic resonance-active marker element, in a further embodiment of the electrode needle according to the invention, can be in the form of a sleeve. A sleeve is easy to produce and to handle.

In a configuration of this embodiment the active electrode encloses an axial portion of the shaft. In that arrangement the sleeve is arranged between the shaft and the active electrode.

In a further embodiment the nuclear magnetic resonance-active marker element is in the form of a wire coil and in particular a helical spring. Particularly if it in the form of a spring, a wire coil can be easily fixed in the interior of the needle by means of a clamping fit. By virtue of its inductance, a wire coil has a nuclear magnetic resonance-active effect even when it does not contain any ferromagnetic material.

In an advantageous development of the embodiment in addition the wire coil can be tuned to the frequency of the nuclear magnetic resonance tomograph. Tuning makes it possible to adapt the intensity of an artefact left behind by the wire coil in the nuclear magnetic resonance tomograph image, to prevailing requirements.

### BRIEF DESCRIPTION OF THE DRAWINGS

Further advantageous properties, features and configurations of the electrode needle according to the invention will be apparent from the detailed description hereinafter of various embodiments with reference to the accompanying drawings.

FIG. 1 is a perspective view of an electrode needle,

FIG. 2 shows the distal end of the electrode needle illustrated in FIG. 1 on an enlarged scale,

FIG. 3 shows a first embodiment of the electrode needle according to the invention in a section taken along its longitudinal axis,

FIG. 4 shows an alternative configuration of the first embodiment in a section taken along the longitudinal axis,

FIG. 5 shows a second embodiment of the electrode needle according to the invention in a section taken along its longitudinal axis,

FIG. 6 shows an alternative configuration of the second embodiment in a section taken along the longitudinal axis,

FIG. 7 shows a third embodiment of the electrode needle according to the invention in a section taken along its longitudinal axis,

FIG. 8 shows a fourth embodiment of the electrode needle according to the invention in a section taken along its longitudinal axis,

FIG. 9 shows an alternative configuration of the fourth embodiment in a section taken along the longitudinal axis, and

FIG. 10 shows a further alternative configuration in a section taken along the longitudinal axis.

### DETAILED DESCRIPTION OF THE PREFERRED EMBODIMENT(S)

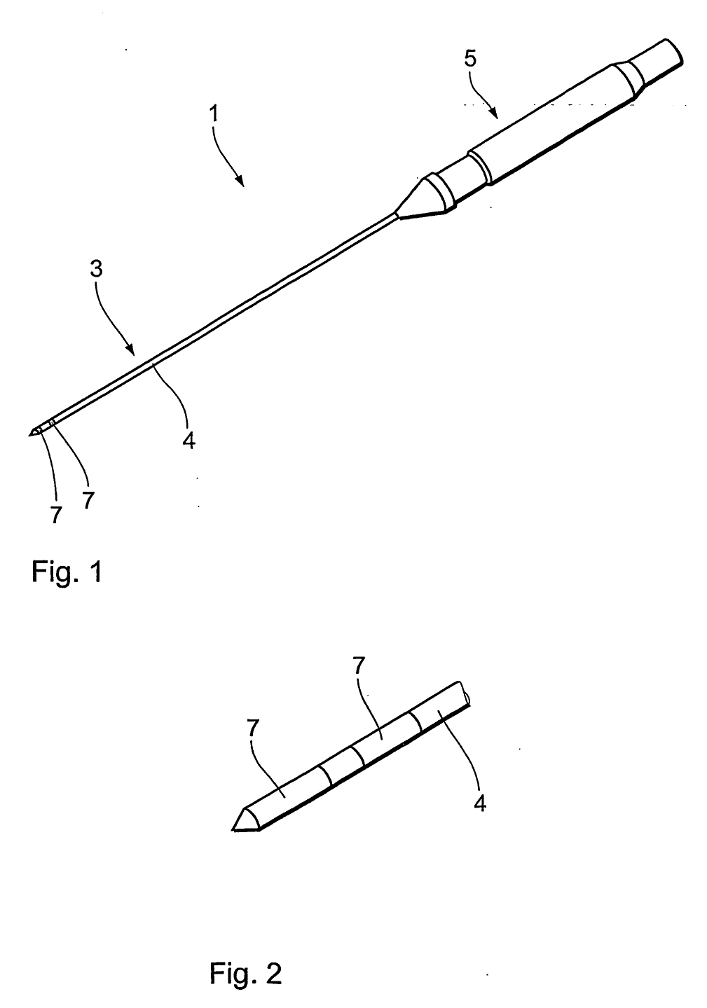

Referring to FIG. 1 shown therein is a perspective view of an electrode needle 1. The electrode needle 1 includes a shaft portion 3 having a shaft 4 which at its distal end has two active electrodes 7. In addition the electrode needle 1 has a gripping portion 5 for handling the needle.

FIG. 2 shows a view on an enlarged scale of the distal end of the shaft 4 with the two active electrodes 7. Admittedly, FIGS. 1 and 2 each show two active electrodes at the distal end of the shaft 4, but, depending on the respective purpose of use (monopolar, bipolar or multipolar treatment) the electrode needle 1 can include any number of active electrodes 7. There is however at least one active electrode 7.

The materials involved for the shaft are body-compatible materials, in particular plastic materials or metals. If it is made from metal, the shaft can be provided in portions in which it is to be insulating with an electrically insulating covering, for example a lacquer or plastic covering. Titanium or a titanium alloy is usually employed for the active electrodes 7 and metallic shafts, by virtue of the good body compatibility thereof. Those materials are nuclear magnetic resonance-compatible by virtue of their paramagnetic properties. In principle other paramagnetic and body-compatible metals may also be considered.

In the present embodiment the shaft 4 of the electrode needle 1 is of a hollow configuration, that is to say it includes a casing 10 which encloses a lumen 8. In that respect the lumen 8 usually servers to accommodate electrode lines for the connection of a high frequency generator (not shown) to the active electrodes 7 and possibly coolant feed lines for cooling the active electrodes 7 in operation.

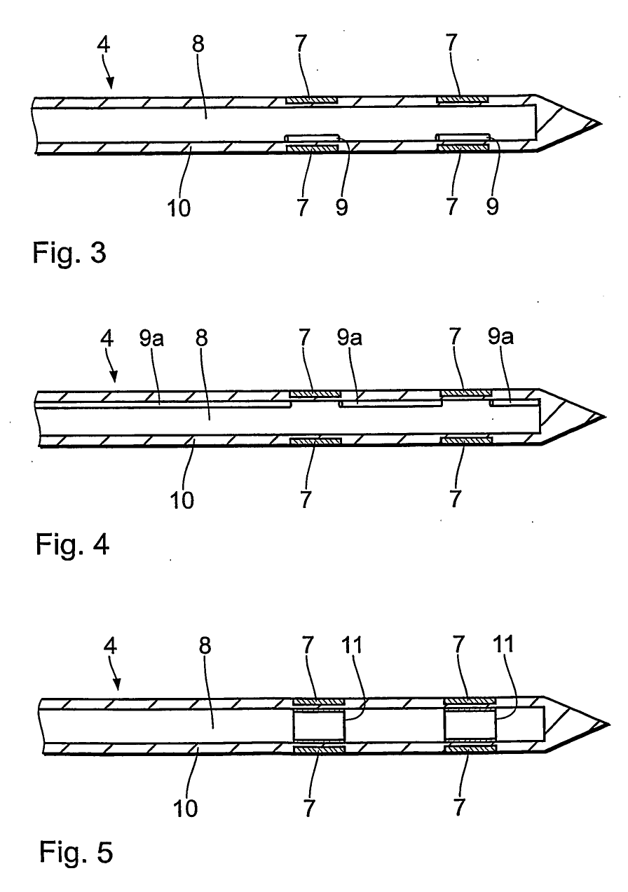

FIG. 3 shows a first embodiment of the electrode needle 1 according to the invention as a section taken along the longitudinal axis of the shaft 4. The shaft 4, the casing 10 and the lumen 8 of the needle can be seen in the sectional view. Arranged at the outside surface of the casing 10 are the active electrodes 7 which annularly surround the casing 10 and extend over a given axial length of the casing. Unlike the situation shown in FIG. 3, the axial length can be different for each active electrode. In the bipolar electrode needle illustrated here the casing 10 is made from an insulating material in order to insulate the two active electrodes 7 from each other and, at the location where the active electrodes 7 are arranged, it is of a wall thickness which is less than the remainder of the shaft 4 so that the electrodes 7 terminate flush with the outside surface of the casing 10.

At the locations where the active electrodes 7 are disposed wire portions 9 of ferromagnetic material, for example steel wire, are arranged at the inside wall of the casing 10. The wire 9 can be for example glued, soldered or spot-welded to the inside surface of the casing 10. Its diameter is advantageously so large that it can be clearly seen in nuclear magnetic resonance-tomographic recording but it is also sufficiently small so that enough room remains in the lumen 8 for further components of the electrode needle 1 which are to be arranged therein.

In the illustrated embodiment the wire 9 is interrupted between the two electrodes 7 so that the two electrodes are marked separately. Alternatively it is also possible for the whole of the active region of the electrode needle 4, which is formed by the two electrodes 7 and the insulation disposed therebetween, to be marked in unitary fashion with a continuous piece of wire. That inexpensive alternative scarcely entails disadvantages as in general it is only the active region of an electrode needle, which is formed jointly by all electrodes, that is of interest.

The wire 9 extends in each case over the entire axial length of an active electrode 7 so that its image in a nuclear magnetic resonance-tomographic recording indicates not only the position but also the length of the active electrode 7.

FIG. 4 shows an alternative configuration of the first embodiment. It differs from the first embodiment in that the nuclear magnetic resonance-active wire 9a extends over the entire length of the shaft 4, with the exception of those regions in which the active electrodes 7 are disposed. In a nuclear magnetic resonance-tomographic recording the result of this is that the portions in which the active electrodes 7 are disposed are delimited by reproductions of the wire 9a. In that sense the nuclear magnetic resonance tomograph image which is obtained by means of the configuration shown in FIG. 4 represents the negative image of that image which was obtained with the configuration from FIG. 3.

FIG. 5 shows a second embodiment of the electrode needle according to the invention. Hereinafter only the differences in relation to the first embodiment will be considered in detail.

Unlike the first embodiment, in this case the nuclear magnetic resonance-active marker element, instead of being in the form of ferromagnetic wire, is applied in the form of a ferromagnetic coating 11 to the inside surface of the casing 10. The coating 11 extends in each case over the axial length of an active electrode 7 over the entire inside periphery of the casing 10. Alternatively the coating can also extend over the entire active region which is formed by both electrodes and the insulation disposed therebetween.

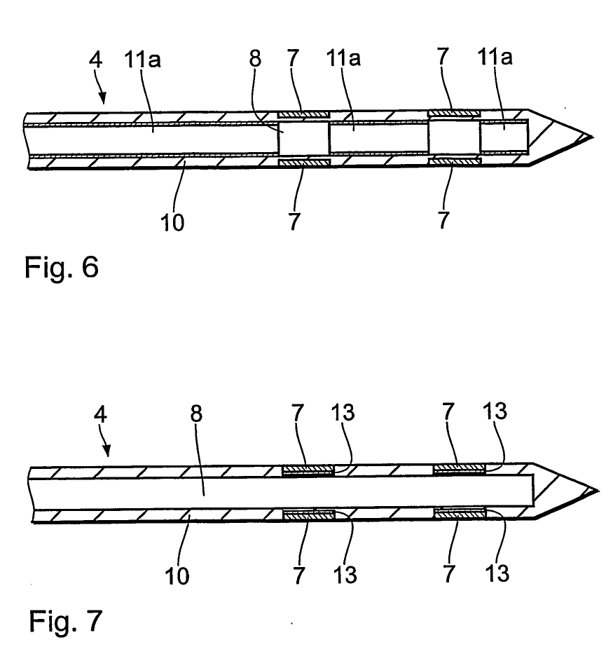

In another alternative configuration (see FIG. 6) the entire inside surface 10 is coated except for those regions in which the active electrodes 7 are disposed. As in the first embodiment this configuration produces so-to-speak a negative image of the active electrodes 7 in a nuclear magnetic resonance-tomographic recording.

FIG. 7 shows a third embodiment of the electrode needle 1 according to the invention. In this embodiment the nuclear magnetic resonance-active material is arranged between the inside surface of the active electrodes 7 and the outside surface of the casing 10.

In the illustrated configuration the nuclear magnetic resonance-active marker element is in the form of a sleeve 13, for example a steel sleeve, which annularly surrounds the outside surface of the casing 10. More advantageously at the locations where the active electrodes 7 are to be fitted the outside surface of the casing 10 has annular grooves which are suitable for accommodating the sleeve 13. The active electrodes 7 are then arranged around the sleeve. The depth of the grooves is preferably so selected that the outside surfaces of the annular electrodes 7 terminate flush with the outside surface of the casing 10. In the other illustrated embodiments it is also advantageous if the outside surface of the active electrodes 7 terminate flush with the outside surface of the casing 10.

Admittedly in the third embodiment the nuclear magnetic resonance-active marker element is in the form of a ferromagnetic sleeve but as an alternative the active electrodes 7 can also be in the form of sleeves, the inner peripheral surface of which is coated with a ferromagnetic material. In a further alternative the active electrodes 7 can also be in the form of sleeves, but in that case the coating can be on the bottom surfaces of the annular grooves.

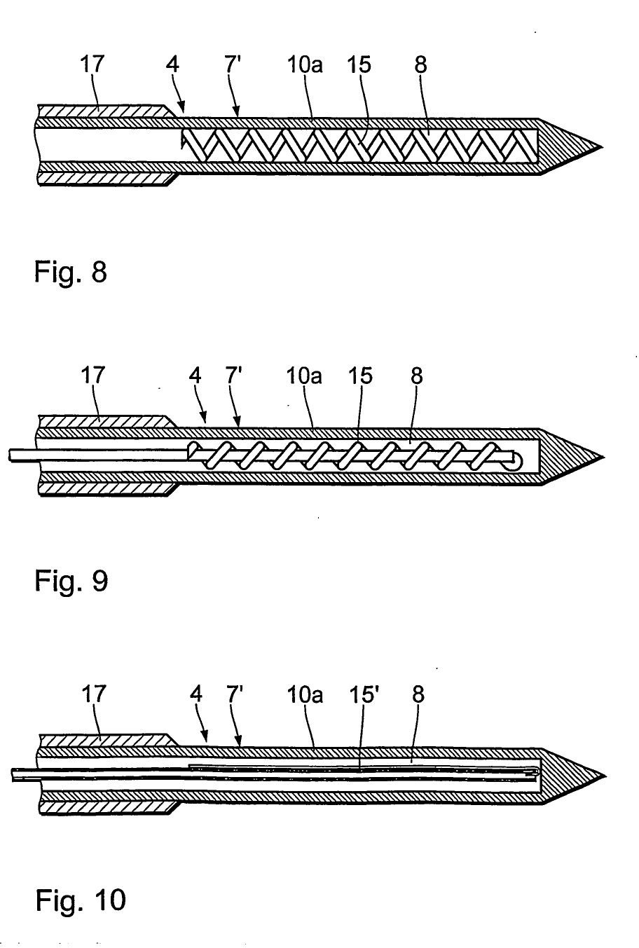

FIG. 8 shows a fourth embodiment of the electrode needle 1 according to the invention. The electrode needle in this embodiment includes a metallic shaft 4, for example of titanium, with a lumen 8 and a casing 10a surrounding the lumen 8. It also includes an insulating jacket 17 which encloses the outside surface of the shaft 4. The distal end of the shaft 4 projects out of the insulating jacket 17 and forms the sole active electrode 7′ of the illustrated electrode needle 1.

Arranged in the interior of the lumen 8 is a wire coil 15 which extends from the distal end of the shaft to the beginning of the insulating jacket 17. It can be fixed to the inside of the shaft 4 for example by soldering, welding, gluing or clamping.

FIG. 9 shows an alternative configuration of that embodiment. A feed line 19 for feeding a coolant is arranged in the lumen 8 of the shaft 4. The wire coil 15 of a nuclear magnetic resonance-active material is wound around the feed line 19 from the distal end of the feed line 19, where it is engaged into the opening of the feed line, to the beginning of the insulating jacket 17. The portion of the wire coil 15 which is engaged into the opening of the feed line 19 is of such a length that, even when the wire coil 15 slips and bears against the distal end of the lumen 8, that portion of the wire coil still partly projects into the opening of the feed line 19 and thus prevents the coil 15 from coming completely loose from the feed line 19.

In both configurations the wire coil 15, in the form of a spring, can also be fixed with a clamping fit in the lumen 8 or around the feed line 19.

Instead of a wire coil alternatively a straight piece of wire of ferromagnetic material can also be engaged into the feed line. That variant is shown in FIG. 10.

1. An electrode needle comprising a shaft and at least one active electrode provided on the shaft, characterized in that the shaft includes a nuclear magnetic resonance-active marker element which is spatially associated with the active electrode.

2. An electrode needle as set forth in claim 1 characterized in that the nuclear magnetic resonance-active marker element extends over the entire axial length of the active electrode.

3. An electrode needle as set forth in claim 1 characterized in that the nuclear magnetic resonance-active marker element extends over the entire axial length of a plurality of active electrodes and the intermediate spaces between them.

4. An electrode needle as set forth in claim 1 characterized in that the nuclear magnetic resonance-active marker element extends over the entire axial length of the shaft with the exception of the axial length of the active electrodes.

5. An electrode needle as set forth in one of claim 1 characterized in that the nuclear magnetic resonance-active marker element is in the form of a wire.

6. An electrode needle as set forth in claim 5 characterized in that the shaft has a lumen and the wire is arranged in the lumen of the shaft.

7. An electrode needle as set forth in claim 6 characterized in that the shaft has a casing with an inside, the casing surrounding the lumen, and the wire is arranged at the inside of the casing.

8. An electrode needle as set forth in one of claim 1 characterized in that the nuclear magnetic resonance-active marker element is in the form of a coating which preferably contains ferromagnetic material.

9. An electrode needle as set forth in claim 8 characterized in that the shaft has a casing with an inside, the casing surrounding the lumen, and the coating being applied to the inside of the casing.

10. An electrode needle as set forth in claim 8 characterized in that the active electrode encloses an axial portion of the shaft, wherein the coating is arranged between the shaft and the active electrode.

11. An electrode needle as set forth in one of claim 1 characterized in that the nuclear magnetic resonance-active marker element is in the form of a sleeve.

12. An electrode needle as set forth in claim 11 characterized in that the active electrode encloses an axial portion of the shaft, wherein the sleeve is arranged between the shaft and the active electrode.

13. An electrode needle as set forth in one of claim 1 characterized in that the nuclear magnetic resonance-active marker element is in the form of a wire coil.

14. An electrode needle as set forth in claim 13 characterized in that the wire coil is tuned to a frequency of the nuclear magnetic resonance tomograph.

15. An electrode needle as set forth in claim 13 characterized in that the wire coil is a helical spring.

16. An electrode needle as set forth in one of claim 1 characterized in that the nuclear magnetic resonance-active marker element is in the form of a straight, nuclear magnetic resonance-active wire preferably containing ferromagnetic material.
